(19) United States
(12) Patent Application Publication
(10) Pub. No.: US 2024/0286821 A1
(43) Pub. Date: Aug. 29, 2024

(54) AEROSOL DISPENSER CONTAINING A HAIRSPRAY COMPOSITION AND A NITROGEN PROPELLANT

(71) Applicant: The Procter & Gamble Company, Cincinnati, OH (US)

(72) Inventors: Jodi Lee BROWN, Cincinnati, OH (US); Mikael Antonio VICENTE, Mason, OH (US); Debra Sue BINSKI, West Chester, OH (US); Casey Robert ALBERTZ, Cincinnati, OH (US)

(21) Appl. No.: 18/423,692

(22) Filed: Jan. 26, 2024

(30) Foreign Application Priority Data
Jan. 30, 2023 (WO) PCT/US2023/061540

(51) Int. Cl. B65D 83/20; A45D 7/00; A45D 34/04

(52) U.S. Cl. CPC B65D 83/205; A45D 7/00; A45D 34/04

(57) ABSTRACT

An aerosol hairspray product with a spray device and an environmentally friendly, pressurizable container. The container includes a container wall that encloses a reservoir for storing a compressed gas propellant and a hairspray composition. The spray properties of the product, including the spray rate, median droplet size, and spray diameter are maintained throughout the lifetime of the product.

### FIELD

An aerosol hairspray product comprising an environmentally friendly, pressurized container that encloses a reservoir for storing a hairspray composition and a compressed gas propellant.

### BACKGROUND

Hairstyling products such as hairsprays can be used to keep hair in place, protect hair from humidity, and create volume. Hairsprays are commonly packaged in an aerosol container, which is under pressure and includes a release valve that is used to emit the pressurized hairspray into the air as a fine mist propelled by a gas propellant. Typically, the propellant is a liquified hydrocarbon propellant. An advantage of a hydrocarbon propellant is that inside the container there is enough pressure to turn the gas into a liquid. As the hairspray product is dispensed, the product level inside the container drops, and more propellant evaporates into the headspace above the product, maintaining an approximately constant pressure, which in turn yields consistent spray properties, such as spray rate and average particle size distribution.

Even though hydrocarbon propellants provide substantial benefits, some consumers would prefer a hairspray product in an aerosol dispenser with a non-hydrocarbon propellant, such as compressed gases, which can include, but are not limited to, compressed air, nitrogen, inert gases, and carbon dioxide. Nitrogen can be especially desirable because it is non-toxic, non-flammable, relatively low in cost and generally inert.

However, it can be difficult to make a consumer acceptable aerosol hairspray product that uses a nitrogen propellant because, unlike liquified hydrocarbons, the nitrogen propellant is always in the vapor state and therefore the pressure in the container is reduced as product is dispensed, making it difficult to dispense the hairspray composition at a consumer acceptable particle size distribution and spray rate over the life of the container. As the pressure inside the container drops, the average particle size distribution increases, eventually releasing globs of hairspray that take too long to dry and can make the hair look dull, limp, and stiff. Eventually, the pressure can be so low that no product is released at all, even if there is product left in the can.

The relationship between various mechanical components of a hairspray product (nozzle, valve, valve stem, dip tubes, etc.) and the rheological properties of the hairspray composition can also impact product performance, including globbing, low spray rate and undesirable spray pattern. Thus, there is need to provide a hairspray product in which the mechanical elements and rheological elements of the hairspray product are tailored to provide suitable product performance.

In addition to good product performance, many consumers also prefer product ingredients and packaging that are environmentally friendly. However, conventional hair styling products continue to be made from virgin materials and/or petrochemical derived materials.

Therefore, there is a need for an aerosol dispenser that contains a hairspray product and a nitrogen gas propellant with consistent spray properties, such as spray rate and average particle size distribution for the life of the container. There is also a need to provide the hairspray product in an environmentally friendly container.

### SUMMARY

Disclosed herein is an aerosol hairspray product comprising an environmentally friendly, pressurized container containing a compressed gas propellant and a hairspray composition. The hairspray composition includes a carrier present at 30% to 98.5% and a hairstyling polymer present at 5% to 8%. The hairspray product also includes a spraying device clinched onto the container. The spraying device includes a valve assembly and a nozzle. The valve assembly includes a housing with internal walls defining a valve chamber. The valve chamber includes a liquid inlet in fluid communication with the hair spray composition and a gas inlet in fluid communication with the compressed gas propellant. The spraying device also includes a valve stem. The proximal end of the valve stem is received in the valve chamber and the distal end of the valve stem projects through a sealed opening in the valve chamber. The valve stem further includes an outlet flow conduit with an outlet aperture at the distal end and a first stem inlet for liquid and a second stem inlet for gas. The housing includes a lip projecting inwardly from the internal walls around the perimeter of the valve stem to form a seal around the valve stem. The valve stem is moveable between a closed position and an open position. In the closed position, the first stem inlet is distal of the lip and the second stem inlet is distal of the sealed opening in the valve chamber, such that neither of the first and second stem inlets is in fluid communication with their respective liquid or gas inlet. In the open position, the first stem inlet is proximal to the lip and in fluid communication with the valve chamber liquid inlet, and the second stem inlet is proximal of the sealed opening in the valve chamber and in fluid communication with the valve chamber gas inlet. When in the open position, a bubble laden flow of the hairspray composition is created in the outlet flow conduit.

### BRIEF DESCRIPTION OF THE DRAWINGS

FIG. 1 shows an exploded diagram of spraying device.

FIG. 2 shows the spraying device clinched on to the container.

FIGS. 3A and 3B schematically illustrate a spraying device in respective closed and open positions.

FIG. 3C is a detail view of part of FIG. 3B, showing the relative positions of an annular lip and a stem gas inlet.

FIGS. 4A and 4B are perspective views of a cap part of a valve housing showing gas flow conduits.

FIG. 5A is a perspective view of a stem forming part of the spraying device.

FIG. 5B is a cross section through the stem of FIG. 5A.

FIG. 6 is a cross-section view of a hairspray product with an actuator

FIG. 7 is a plan view of a hairspray product with an overcap.

### DETAILED DESCRIPTION

Many consumers would be interested in an aerosol hairspray product with a non-hydrocarbon propellant, such as compressed gas, that has excellent spray properties that are maintained throughout the life of the product (i.e. from 100% product (full can) to when 25% of the product was left in the can (end of can)). Compressed gas including air, nitrogen, carbon dioxide, and other inert gases can be desirable because they are non-toxic and non-flammable. In some examples, nitrogen gas can be preferred.

Hydrocarbon propellants are commonly used in aerosol hairspray products. These propellants are present (in the aerosol spray device) both a gas phase and a liquid phase, which is miscible with the liquid hairspray composition. Examples of hydrocarbon propellants can include butane, propane, dimethyl ether, isobutane, 1,1-Difluoroethane, or mixture thereof. On discharge, the gas phase propellant “propels” the liquid in container (including dissolved, liquid phase propellant through the nozzle).

One reason hydrocarbon propellants are popular is because they are capable of producing finer sprays than compressed gas aerosols because a large proportion of the liquefied gas “flash vaporizes” during discharge of liquid from the aerosol spray device and this rapid expansion gives rise to a fine spray. It is difficult to get these fine sprays using compressed air.

It was found that an aerosol hairspray product with an aerosol dispenser with a valve assembly, as described herein, in combination with ethanol-based and/or ethanol-free hairspray compositions and a compressed air propellant, such as nitrogen, can have consumer preferred spray properties (such as spray rate, particle size, spray diameter) throughout the life of the product to when the container contains 25% or less, by weight, of the hairspray composition. Providing a suitable spray rate and Dv50 droplet size deliver sufficient hairspray composition to keep hair in place, provide desirable hold, and/or create volume, while having a non-sticky and natural hair look and feel. Since ethanol-free hairsprays typically contain high levels of water (e.g., 30-60%), as opposed to volatile ethanol which flashes off, too much hairspray composition can be delivered to the hair, causing it to become overwetted. Not only does overwetted hair longer to dry, which is undesirable, the hairspray composition can disrupt the internal ionic interactions of the hair, allowing the hair to relax and lose the desired style. The dispensing problems can be exacerbated as propellant is expelled from the can throughout the lifetime of the product.

The hairspray product described herein is an aerosol hairspray product and does not include mousse products or any pump spray products. The aerosol hairspray product can include an ethanol-based hairspray composition or an ethanol-free (e.g., water-based) hairspray composition.

Table 1, below, shows desired spray properties for ethanol-based hairspray products and ethanol-free hairspray products. In particular, these properties are desired at the initial spray when the container is full and when there is 25% or less of the hair spray composition remaining in the container. Accordingly, the spray properties in Table 1, collectively or individually, should vary by no more than 30% (e.g., no more than 25%, 20%, 15%, or even 10%) between the initial/full container measurements and when 25% of the product remains. In some aspects, it may be desirable for the spray droplet size to vary by no more than 30 μm (e.g., no more than 25 μm, 20 μm, 15 μm, or even 10 μm). The spray properties shown in Table 1 can be determined according to their respective test methods described in more detail below.

TABLE 1
Spray Properties

| | | |
|---|---|---|
| Ethanol- | Spray Rate | 0.3 g/sec to 1.0 g/sec |
| based | Dv50 | 40 μm to 90 μm |
| Hairspray | Droplet Size | |
| | Spray | 5 cm to 15.25 cm |
| | Diameter | |
| Ethanol-free | Spray Rate | 0.3 g/sec to 0.6 g/sec |
| Hairspray | Dv50 | 40 μm to 80 μm |
| | Droplet Size | |
| | Spray | 5 cm to 15.25 cm |
| | Diameter | |

“About” modifies a particular value by referring to a range of plus or minus 20% or less of the stated value (e.g., plus or minus 15% or less, 10% or less, or even 5% or less).

“Hair” means mammalian hair including scalp hair, facial hair and body hair, more preferably hair on the human head and scalp. “Hair shaft” means an individual hair strand and may be used interchangeably with the term “hair.”

“Life of the product” means the time from when the container contains 100% of the hairspray composition initially placed in the container to when the container contains 25% or less of the hairspray composition initially placed in the container.

“Molecular weight” or “M.Wt.” refers to the weight average molecular weight unless otherwise stated. Molecular weight can be determined according to the industry standard method of gel permeation chromatography (“GPC”).

“Substantially free of” means 2% or less (e.g., 1% or less, 0.5% or less, or 0.1% or less) of a stated ingredient. “Free of” means no detectable amount of the stated ingredient or thing.

“Water-soluble” means a material is sufficiently soluble in water to form a single-phase solution to the naked eye at a concentration of 0.1% by weight of the material in water at 25° C. It may be necessary to adjust the pH of the mixture or fully neutralize the mixture after addition of the material to water to achieve the water solubility. These methods are known, for example, in the water-soluble hairstyling polymer applications industry and are typically instructed with the supplied material sample. Water-solubility is typically measured by the following protocol: 0.1% by weight of the material is added to distilled water at 25° C. and the pH adjusted/neutraliser added as needed. This is stirred vigorously on a magnetic stirrer set at 600 rpm, for 30 minutes. The solution is then allowed to settle for 1 hour and the number of phases observed by the naked eye. For example, where any solid material can be seen in an otherwise single-phase solution, then this is considered to be two phases.

All percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated.

Unless otherwise indicated, all measurements are understood to be made at 25° C. and at ambient conditions, where “ambient conditions” means conditions under one atmosphere of pressure and at 50% relative humidity. All such weights as they pertain to listed ingredients are based on the active level and do not include carriers or by-products that may be included in commercially available materials, unless otherwise specified.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Pressurizable Container

The aerosol hairspray product comprises a pressurizable container. The type of container is not particularly limited and may include cans, bottles or other suitable container types known for use with hairspray products. The container may be formed from any suitable material including metal, plastic, pulp, glass and combinations thereof. The pressure inside the reservoir can be measured with a pressure gauge (GCAS #60001439). The internal compressed gas (e.g., nitrogen) pressure can be determined based on the DOT 2Q regulations for an aerosol can. In some embodiments, the pressure inside the container can be 100 to 159 PSIG (e.g., 110 to 150 PSIG, 120 to 140 PSIG, or even 130 to 135 PSIG), at 70° F.

### Spraying Device

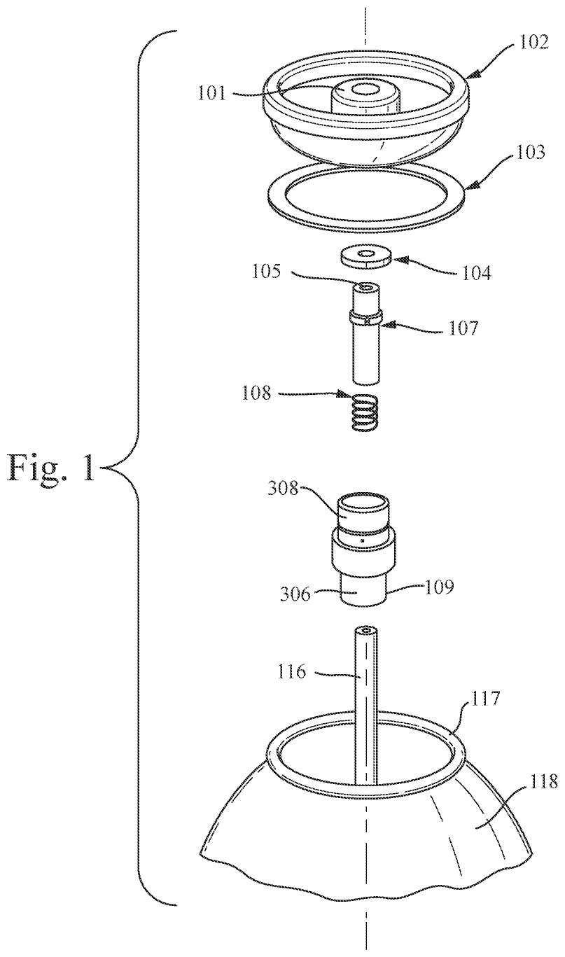

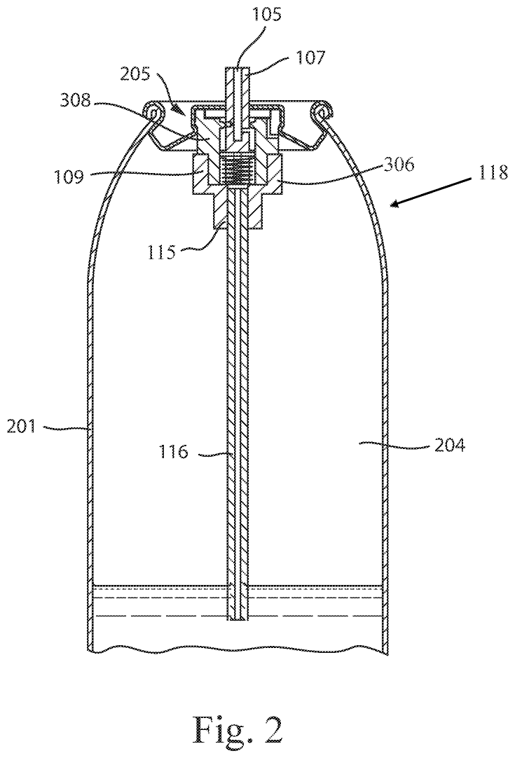

FIG. 1 is an exploded view of an exemplary spraying device for dispensing a hairspray composition, and FIG. 2 shows the spraying device clinched on to a container. The aerosol hairspray product includes a spraying device attached to a container 118 for dispensing the hairspray composition from the reservoir 204 of the container 118. The spraying device includes a valve assembly 205 and a nozzle. The valve assembly 205 includes a valve housing 109, a stem 107 and a spring 108. The housing 109 includes a lower cup portion 306 and an upper cap portion 308. In this example, the valve assembly 205 is in liquid communication with the hairspray composition in the reservoir 204.

The valve housing 109 can have a valve tailpiece 115, and wherein the valve tailpiece 115 has an orifice, which receives a dip tube 116 that is capable of being in fluid communication with the hairspray in reservoir 204. The valve tailpiece 115 orifice (which receives the dip tube 116) can have an inner diameter of 0.762 mm to 1.778 mm (e.g., 0.889 mm to 1.651 mm, 1.016 mm to 1.524 mm, 1.143 mm to 1.397 mm, or 0.889 mm to 1.143 mm).

The stem 107 has a stem orifice 105, which acts as an outlet for the contents of the container (i.e., the hairspray composition and the propellant). The stem orifice 105 can have an inner diameter of 0.127 mm to 0.635 mm (e.g., 0.203 mm to 0.340 mm, or 0.279 mm to 0.356 mm). The valve assembly 205 may include a stem gasket 104 that seals against the seat for the stem gasket on the stem 107 and, optionally, covers a side hole in the stem that leads to the stem orifice 105. In some embodiments, the stem gasket 104 may be pre-assembled onto the stem 107. The stem 107 and valve housing 109 may be made of polyphenylene sulfone.

A spring 108 may be prefitted to the base of the stem moulding. The spring may be made of a material that is resistant to corrosion or oxidation (e.g., plastic or stainless steel). The mounting cup 102 may be prefitted with a cup gasket 103 to form a gas-tight seal against the container curl 117 when the assembled valve is clinched to the container. The subassemblies described above are crimped together using a pedestal crimping tool to make the fully assembled valve assembly 205. The stem gasket 104 is compressed by ˜50% in thickness by the crimping procedure, and the pedestal of the mounting cup 102 is deformed during crimping to engage and retain a surface of the valve housing 109. A dip tube 116 (e.g., made of polyethylene) is push-fitted into a sealing engagement with the tailpiece 115 to complete the valve assembly 205.

The hairspray composition and compressed gas propellant are placed in the container 118 before the valve assembly 205 and dip tube 116 are clinched onto the container curl 117 by use of conventional clinching equipment to make a gas-tight seal between the valve assembly 205 and the container 118. The container 118 is then pressurized to the desired working pressure by gassing through the stem orifice 105. When the stem 107 is depressed by more than approximately 1 mm through application of external force (e.g., to an actuator coupled to the stem) the stem gasket 104 deforms away from the side hole in the stem gasket seat 106 area, opening a path between the container 118 and the external environment. When the external force is released, the spring 108 returns the stem to its fully closed position.

As shown in FIG. 2, the valve assembly 205 is clinched on to the top of the container 118 after filling with the hairspray composition. The stem orifice 105 is the outlet of the stem 107. A container wall 201 defines a reservoir 204 for storing a hairspray composition and a compressed gas propellant. Activation of the valve assembly 205 is achieved by through application of an external force to depress the engaged stem 107 and thereby release the hairspray composition and propellant into the external environment via a nozzle (not shown). The valve assembly 205 comprises a housing 109 which mounts the dip tube 116 and which admits propellant gas from reservoir 204 into the flow of hairspray composition which rises up the dip tube 116 on operation of valve assembly 205 opening.

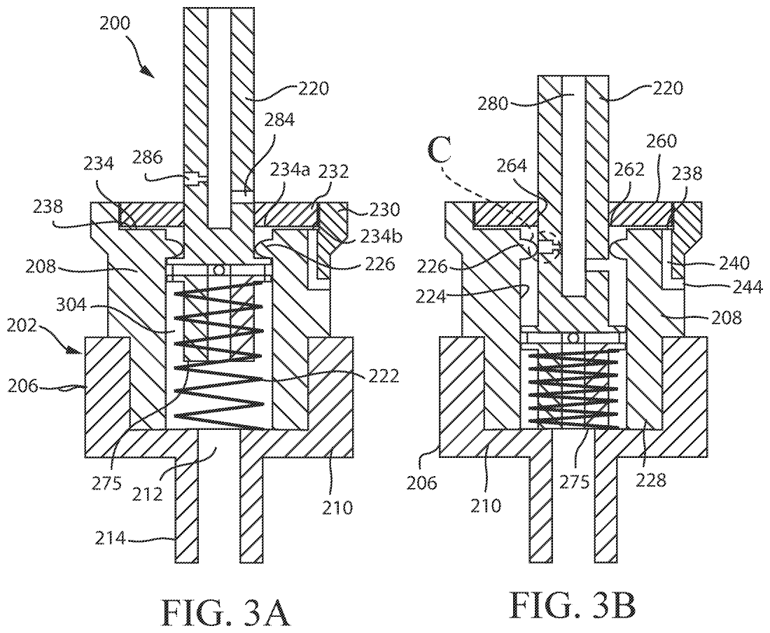

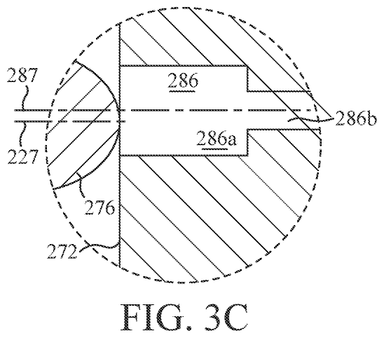

FIGS. 3A and 3B illustrate another example of a valve assembly. FIG. 3C is a magnified view of element C of FIG. 3B. The valve assembly 200 shown in FIGS. 3A and 3B can be incorporated into the aerosol spray devices of FIGS. 1 and 2. The valve assembly 200 includes a housing 202 with internal walls defining a valve chamber 304, and a valve stem 220. The housing 202 is formed of two portions: a lower, cup portion 206; and an upper, cap portion 208. The valve assembly 200 can be crimped in place at the top of a container (e.g., container 118 in FIGS. 1 and 2), with a distal portion of the valve stem 220 projecting from the top of the container for connection to an actuator.

The cup portion 206 has a lower wall 210 with an aperture 212 therethrough. A tubular spigot 214 extends from the lower wall 210. A dip tube (not shown) can be connected to the tubular spigot 214, typically by means of an enlarged lower end, the dip tube extending to the base of the container to which the valve assembly 200 is fitted. It will be appreciated that the lower region of a container to which the valve assembly 200 is fitted is in fluid communication with the valve chamber 304 via the dip tube, spigot 214 and aperture 212 (which provides a liquid inlet for the valve chamber).

As shown in FIG. 3C, the stem gas inlet 286 is moved to a position in which it is marginally offset distally from the lip 226, i.e., a central axis 287 of the stem gas inlet 286 is just above the centerline 227 of the lip 226. This allows not only gas from the valve chamber gas inlet 234a to enter the stem gas inlet 286, but also a small amount of liquid from the valve chamber liquid inlet 212 too.

In some embodiments, the stem gas inlet 286 is stepped, having an outer portion 286a (opening to the stem surface 272) with a larger diameter than an inner portion 286b (opening to the outlet conduit 280). Alternatively, the stem gas inlet 286 may have a conical cross-section, tapering from a larger outer portion to a smaller inner portion. In construction of the valve assembly 200, the total cross-sectional area of the gas bleed passageways 240, 238, 234, 286 should not be so large that excessive gas is bled into the outlet conduit 280 such that the container is depleted of pressurized propellant before all of the hairspray composition has been discharged. The total cross-sectional area of the gas bleed inlet passageways may be equivalent to that of a singular, circular section inlet with a diameter of 0.15-0.8 mm.

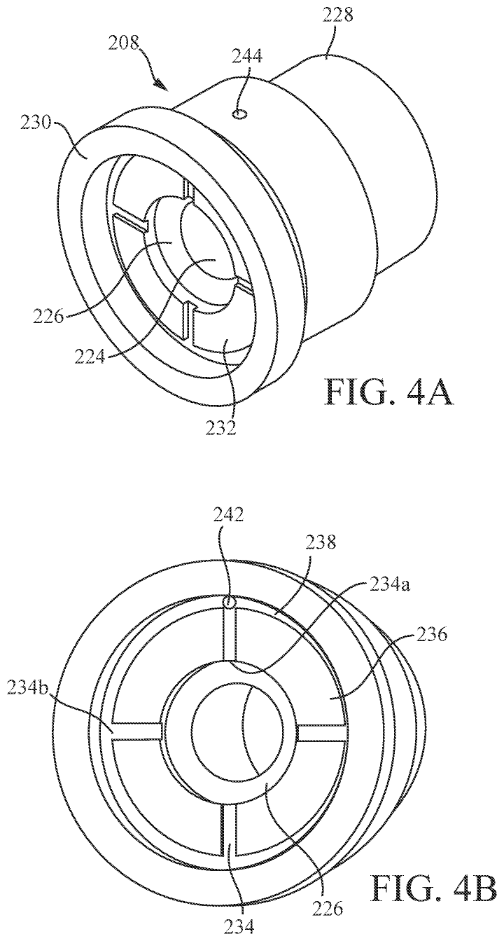

FIG. 4A illustrates an example of a cap portion 208 from FIGS. 3A and 3B. As shown in FIG. 4A, the cap portion 208 has a generally cylindrical inner wall 224 from which a lip 226 projects inwardly at the upper end thereof. The lower end 228 of the cap portion has a narrower outer diameter so as to fit with an interference inside the cup portion 206. At the upper end of the cap portion 208, an annular rim 230, together with an upper surface 232, defines a shelf within which an annular sealing gasket 260 sits.

FIG. 4B shows a plurality of radial grooves 234 defined between corresponding radial ribs 236 on the upper surface 232. Inner ends 234a of the grooves 234 open into the upper end of the valve chamber, above the lip 226. Outer ends 234b of the grooves 234 open into a circumferential groove 238, which circumscribes the upper surface 232 just inside the rim 230. The lower and side surfaces of the respective grooves 234, 238 are formed by the cup portion itself, whereas the upper surfaces thereof are formed by the lower surface 262 of the gasket 260.

A conduit 240 is formed through the cap portion 208, with an upper end opening into the circumferential groove 238 via a hole 242, and with a lower end exiting the side of the cup portion 206 via a hole 244 in the outer surface thereof. It will be appreciated that the head space of a container to which the valve assembly 200 is fitted is in communication with the valve chamber 304 via the conduit 240, circumferential groove 238 and radial grooves 234 (which together provide a gas inlet for the valve chamber).

The valve stem 220 is generally cylindrical, having an outer surface 272 with a diameter equal to the inner diameter of the lip 226, such that the lip 226 forms a seal around the perimeter of the valve stem 220. A proximal end 274 of the valve stem is received in the valve chamber 304 and a distal end 276 projects through the center 264 of the annular sealing gasket 260, which is dimensioned to seal against the outer surface 272 of the valve stem 220. The lower surface 262 of the gasket 260 defines the top of the valve chamber 304.

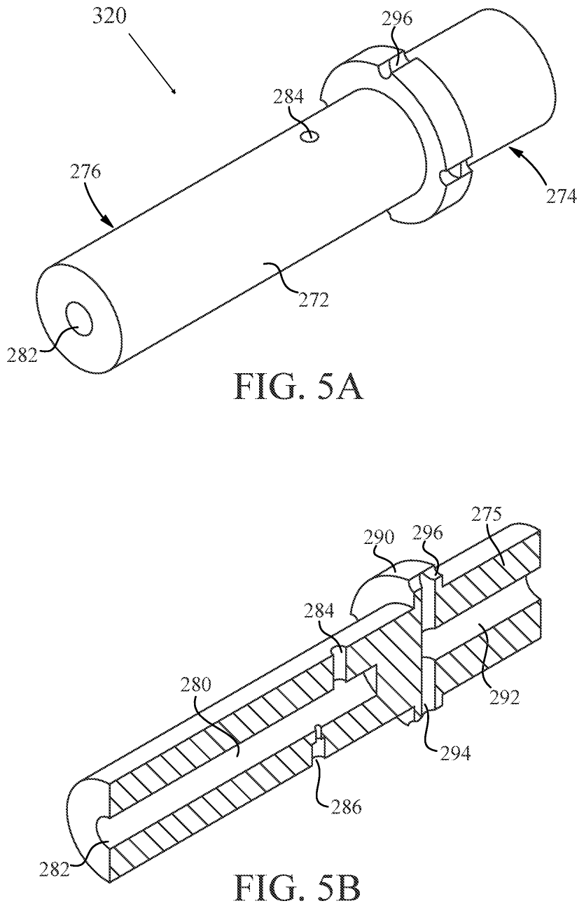

FIGS. 5A and 5B illustrate an exemplary valve stem 320 for use in the present hairspray product. The valve stem 320 includes an outlet flow conduit 280 with an orifice 282 at the distal end 276 and, more proximally, at least one first stem inlet 284 for liquid and at least one second stem inlet 286 for gas. As illustrated, there is a single stem inlet 284 for liquid and a single stem inlet 286 for gas, and they are positioned roughly in the middle of the valve stem, with the gas inlet 286 being slightly distal of the liquid inlet 284. It will be understood that alternative arrangements are envisaged. For example, there could be multiple liquid inlets 284 and/or multiple gas inlets 286; the inlets 284, 286 could be located more proximally or more distally than shown; and the axial separation between the respective liquid and gas inlets could be greater than shown.

As shown in FIGS. 5A and 5B, towards the proximal end 274 of the valve stem 220, an enlarged shoulder portion 290 projects radially from the cylindrical valve stem 220. The diameter of the shoulder 290 is substantially equal to that of the valve chamber 304. A bore 292 runs centrally from the proximal end face 275 valve stem 220 to the shoulder portion 290. Four conduits 294 extend radially within the shoulder portion 290 from the center, where they open into the bore 292, to the outside. At the outer ends, the radial conduits 294 open into respective axial grooves 296 in the outer surface of the shoulder 290 that run parallel to the bore 292 and to the outlet conduit 280.

As shown in the drawings, the valve stem 220 is biased upwardly of the valve assembly (and thus of the aerosol device) by means of a coil spring 222. Lower end of coil spring 222 locates around the aperture 212 of the cup portion 206 of the housing 202. In the closed valve position, as shown in FIG. 3A, the shoulder 290 abuts against the lip 226 under the force of the spring 222, and the flow channel defined by the bore 292, radial conduits 294 and axial grooves 296 is blocked by virtue of the tops of the axial grooves 296 abutting against the underside of the lip 226. Furthermore, the liquid inlet 284 is more distal than the sealing gasket 260. Accordingly, there is no fluid communication between the valve chamber liquid inlet 212 and the outlet conduit 280. There is also no fluid communication between the valve chamber gas inlet 234a and the outlet conduit 280, because the gas inlet 286 is also more distal than the sealing gasket 260, which hermetically seals against the outer surface 272 of the valve stem.

The abutment of the shoulder 290 against the lip 226 acts as an upper limit stop, preventing the valve stem 220 from being urged further out of the valve housing 202.

When the valve stem is moved to the open position, as shown in FIG. 3B, the stem liquid inlet 284 is moved below (i.e. proximal of) the lip 226 so as to be in fluid communication with the valve chamber liquid inlet 212 via the flow channel defined by the bore 292, radial conduits 294 and axial grooves 296 through the stem shoulder portion 290. Also, the stem gas inlet 286 is moved below (i.e. proximal of) the sealing gasket 260 to a position at the upper end of the valve chamber 304 in fluid communication with the valve chamber gas inlet 234a. At least a part of the stem gas inlet 286 must be open to the upper portion of the valve chamber 304 (i.e. the portion above the lip 226). Abutment of the bottom face 275 of the valve stem 220 against the lower wall 210 of the cup portion 206 defines a lower limit stop.

Thus, to operate the device, an actuator cap is depressed so that the valve stem 220 moves downwardly against the bias of spring 222 from the closed position to the open position. As a result, the liquid and gas stem inlets 284, 286 are displaced past the gasket 260 and brought into respective fluid communication with liquid hairspray composition from the container 2 and compressed gas from the head space.

Compressed gas can now flow into the outlet conduit 280 by passage through the hole 244 in the outer surface of the cap portion 208, the conduit 240, the hole 242, the circumferential groove 238 and radial grooves 234, and through the stem gas inlet 286.

Hairspray composition can now flow into the upper portion of the valve chamber 304 by passage upwardly along the dip tube 20, through the inlet 212, the bore 292, the radial conduits 294 and the axial grooves 296. Hairspray composition introduced into the upper portion of the valve chamber 304 passes via stem liquid inlet 284 into flow conduit 280 where it is mixed with the compressed gas bled through the stem gas inlet 286. A bubble laden flow of homogeneous bubbles with similar diameters (Dv50) and without significant coalescence or stratification is formed in the outlet flow conduit 280. That bubbly flow can then flow, preferably undisturbed, through the stem orifice and actuator.

A nonlimiting example of a spraying device suitable for use with the present hairspray product is an Ecovalve® device provided by Salvalco, York, GB. Such a valve is disclosed in U.S. Pat. No. 10,071,849.

### Actuator

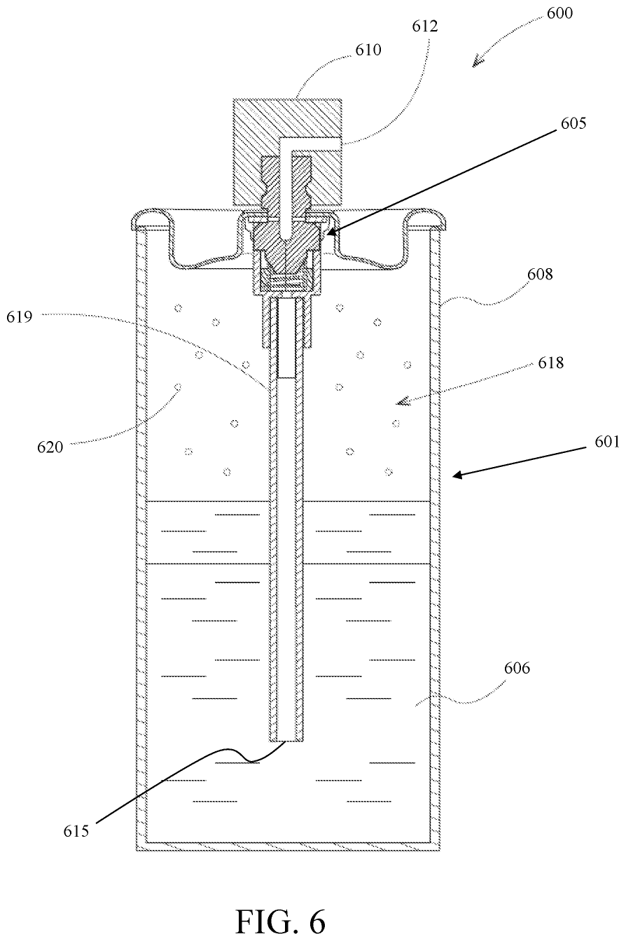

FIG. 6 illustrates an exemplary hairspray product 600 that includes a container 601, a spraying device 605, and a spray actuator 610. As illustrated in FIG. 6, a container wall 608 encloses a reservoir 618 containing a compressed gas propellant 620 and a hairspray composition 606. The spraying device 605 can be one of the spraying devices described herein. A dip tube 619 extends into the reservoir 618 and provides fluid communication between the reservoir and the valve assembly of the spray device 605. The valve assembly and valve stem of the spray device 605 are configured to provide fluid communication between the dip tube and the spray actuator 610. When sufficient force is applied to the spray actuator 610, the hairspray composition 606 travels up through the dip tube orifice 615 and the dip tube 619, and out of the actuator outlet orifice 612.

In some embodiments, it may be desirable to use a button-type actuator to provide a short flow path for the hairspray composition to travel through the actuator. Additionally or alternatively, it may be desirable to provide spray-through cap type of actuator to remove the need for a separate overcap. While the type of actuator is not particularly limited, it can be important to configure the actuator to function with (e.g., be seated on) a vertically configured valve assembly and/or valve stem, as illustrated in FIGS. 1 and 2. In this configuration, the valve is actuated by applying a downwardly directed actuating force, rather than a side directed force, for example, as used in a tilt valve configuration.

In some instances, it may be desirable to configure the valve stem and the actuator to have a ratio of valve stem orifice area to actuator outlet orifice area of less than 10 (e.g., less than 9, 8, 7, or even less than 5). If an actuator outlet insert is optionally used, then the ratio of valve stem orifice area to actuator insert area can be less than 10. It has been found that there can be a noticeable difference in the ability of a valve assembly and actuator nozzle to atomize a hairspray composition. Without intending to be bound by any theory, it is believed that by keeping the area of the actuator outlet orifice closer to the area of the valve stem orifice, one avoids a rush of hairspray composition thru the actuator that can become “bottlenecked” at the outlet. If the hairspray composition bottlenecks at the outlet orifice, then it will lose the necessary energy for atomization. By valve stem orifice, it is meant the orifice though which the hairspray composition passes. Some spray devices may have a second orifice through which only the compressed gas passes.

### Overcap

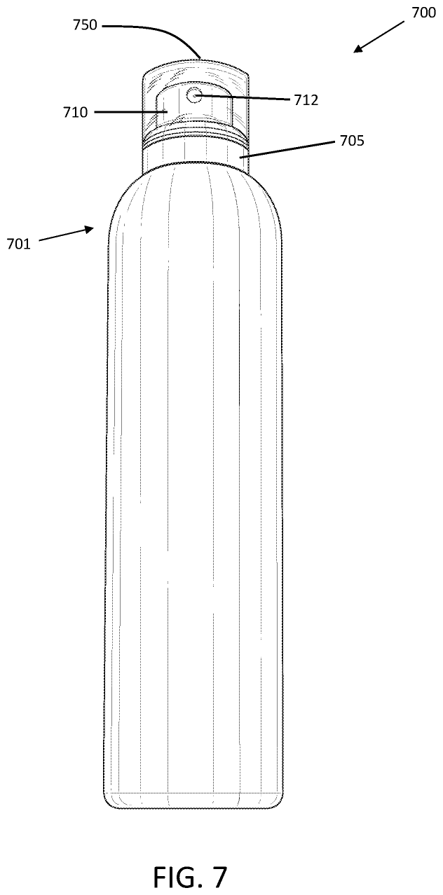

FIG. 7 illustrates an example of a hairspray product 700 with an overcap 750 to prevent inadvertent discharge of the hairspray composition, provide tamper resistance or act as a tamper evident seal, protect the actuator from damage, and/or harmonize the aesthetic appeal of the hairspray product. The overcap 750 covers a top portion of the hair spray product 700 (e.g., spray actuator 710, valve stem and valve assembly). The overcap 750 may be releasably attached to the container 701 or container neck 705 by way of an outwardly protruding ridge, which circumscribes the interior lower edge of the overcap and interacts with a bead or seam that circumscribes a top portion of the container 701. When the overcap 750 is placed onto the top portion of the container 701, downward pressure can be applied to the overcap 750 causing the ridge to ride over an outer edge of the seam and lock under a ledge defined by a lower surface of the seam. The overcap 750 may be transparent, translucent, opaque, colored or colorless, as desired. As shown in FIG. 7, the overcap 750 colorless and transparent such that the actuator outlet orifice 712 is generally visible to a user.

The container, spraying device, actuator and/or overcap may be made from any suitable material desired. Particularly suitable materials for making these features include recyclable, recycled and/or sustainably sourced materials. For example, one or more of the container, spraying device, actuator and/or overcap may be constructed from a material that includes 10% to 100% (e.g., 20%, 30%, 40%, 50%, 60%, 70%, 80%, 90% or even 100%) recyclable, recycled or sustainably sourced material. Some non-limiting examples of recyclable materials include metal, glass, plastic and paper. Some non-limiting examples of recycled materials include plastics made from post-consumer recycled (PCR) resin, recycled aluminum and recycled glass. Some non-limiting examples of sustainably sourced container materials include bio-based plastics, which are polymeric materials made from renewable carbon feedstocks such as plants (e.g., soybeans, corn, or sugar cane). Bio-based plastics suitable for use herein have a Percent Modern Carbon value of at least 10% (e.g., 20%, 30%, 40%, 50%, 60%, 70%, 80%, 90% or even 100%), according to ASTM D6566-10 method B. Further examples and descriptions of bio-based plastics and Percent Modern Carbon can be found in U.S. Pat. Nos. 11,433,158 and 10,166,312.

### Hairspray Composition and Compressed Gas Propellant

The aerosol hairspray product herein includes a hairspray composition and a compressed gas propellant stored in a reservoir of the container. The compressed gas propellant and hairspray composition may be stored in the same or separate compartments. The reservoir can contain 50% to 65% hairspray composition (e.g., 55% to 60%), by volume. The reservoir can include 35% to 70% propellant, (e.g., 40% to 60%, 45% to 55% or 45% to 50%), by volume. As used herein, unless otherwise stated, details of the hairspray composition and propellant refer to the composition before it is placed into the can.

The hair spray composition is dispensed as a multitude of droplets having an average particle size distribution (Dv50) of 40 microns to 100 microns, (e.g., 40 microns to 90 microns, 60 microns to 80 microns), as determined by the Particle Size Distribution Test, described below. The spray delivery rate for ethanol-based hairsprays can be
0.2 g/s to 0.9 g/s (e.g., 0.25 g/s to 0.8 g/s, 0.28 g/s to 0.75 g/s, or 0.3 g/s to 0.7 g/s), as determined by the Delivery Rate Test Method. The spray delivery rate for ethanol-free hairsprays can be 0.2 g/s to 0.6 g/s (e.g., 0.25 g/s to 0.55 g/s or 0.3 g/s to 0.5 g/s), as determined by the Delivery Rate Test Method.

The kinematic viscosity of an ethanol-free hairspray composition herein, without propellant, can be 0.5 cSt to 5.5 cSt (e.g., 1 to 5 cSt, 1.25 to 4.5 cSt, 1.5 to 4.0 cSt, 1.75 to 3.5 cSt, 1.8 to 3 cSt, or even 2.0 to 2.5 cSt). The kinematic viscosity of an ethanol-based hairspray composition herein, without propellant, can be 1 to 20 cSt (e.g., 3 to 18 cSt, or 5 to 15 cSt).

The hairspray composition can include 1.5% to 10% hairstyling polymer, by weight of the hairspray composition. The amount of hairstyling polymer is important in balancing hold performance and on-hair wetness. The amount of hairstyling polymer drives the hold performance but is limited by a maximum sprayable viscosity. The hairspray composition can include 2% to 8% (e.g., 3% to 7% or 3.5% to 6%) hairstyling polymer, by weight of the hairspray composition. These amounts may be the total amount of hairstyling polymer in the hairspray composition.

The hairstyling polymer or mixture of hairstyling polymers can be water-soluble hairstyling polymers and/or ethanol/alcohol-soluble hairstyling polymers that can provide a viscosity of 6 cSt or less as measured before the addition of propellant. This hairspray composition containing soluble hairstyling polymer(s) is then pressurized in a can with a gas propellant. In some examples, a user may shake the can prior to dispensing in order to mix the hairspray composition with the hairstyling polymer and the propellant.

The hairstyling polymer may be any water-soluble or alcohol-soluble film-forming polymer or mixture of such polymers. This includes homopolymers or copolymers of natural or synthetic origin having functionality rendering the polymers water-soluble such as hydroxyl, amine, amide or carboxyl groups.

The soluble hairstyling polymers when diluted at the range claimed, can form transparent or semi-transparent stable solutions. Depending on the specific polymer type, it may be necessary to adjust the pH of the formulation or to neutralize the formulation after addition of the polymer to water to achieve water solubility. The hairstyling polymer may be classified into two types, (totally) synthetic polymers and natural products together with their chemically modified derivatives and further can be grouped into three main headings; naturally occurring, semi-synthetic and completely synthetic polymers. The hairstyling polymer can be selected from the group consisting of: cationic hairstyling polymers, anionic hairstyling polymers, nonionic hairstyling polymers, and amphoteric hairstyling polymers. The molecular weight of the hairstyling polymers should be such that the hairspray composition without propellant meets the viscosity requirement range specified. The hairstyling polymers can be linear or branched.

The hairstyling polymer may be a cationic hairstyling polymer, anionic hair styling polymers, non-ionic hairstyling polymer, an amphoteric hairstyling polymer or mixtures thereof. The cationic hairstyling polymer can be selected from the group consisting of: quaternized acrylates or methacrylates; quaternary homopolymers or copolymers of vinylimidazole; homopolymers or copolymers comprising a quaternary dimethdiallyl ammonium chloride; non-cellulosic cationic polysaccharides; cationic cellulose derivatives; chitosans and derivatives thereof; and mixtures thereof.

The hairstyling polymer can be an anionic hairstyling polymer or a mixture of anionic hairstyling polymers. The anionic hairstyling polymer can be selected from those comprising groups derived from carboxylic or sulfonic acids. Copolymers containing acid units are generally used in their partially or totally neutralized form, more preferably totally neutralized. The anionic hairstyling polymer can comprises\: (a) at least one monomer derived from a carboxylic acid such as acrylic acid, or methacrylic acid or crotonic acid or their salts, or C4-C8 monounsaturated polycarboxylic acids or anhydrides (e.g., maleic, furamic, itaconic acids and their anhydrides) and (b) one or more monomers selected from the group consisting of: esters of acrylic acid and/or methacrylic acid (e.g., C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g., poly(ethyleneglycol)acrylate); hydroxyesters acrylate (e.g., hydroxyethyl methacrylate); acrylamides, methacrylamides which may or may not be substituted on the nitrogen by lower alkyl groups (C1-C4); N-alkylated acrylamide (e.g., N-tertbutylacrylamide); hydroxyalkylated acrylamide; amino alkylated acrylamide (e.g., dimethyl amino propyl methacrylamide); alkylacrylamine (e.g., tert-butylamino-ethyl methacrylate, dimethyl aminoethyl methacrylate); alkylether acrylate (e.g., 2-ethoxyethyl acrylate); monoethylenic monomer such as ethylene, styrene; vinyl esters (e.g., vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; vinyllactams such as vinylpyrrolidone or vinylcapro lactam; alkyl malcimide, hydroxyalkyl malcimide (e.g., Ethyl/Ethanol Maleimide). When present the anhydride functions of these polymers can optionally be monoesterified or monoamidated. The anionic hairstyling polymer can comprise monomers derived from a sulfonic acid. Anionic polymers can comprise: (a) at least one monomer derived from a sulfonic acid such as vinylsulfonic, styrenesulfonic, naphthalenesulfonic, acrylalkyl sulfonic, acrylamidoalkylsulfonic acid or their salts and (b) one or more monomers selected from the group consisting of: esters of acrylic acid and/or methacrylic acid (e.g., C1-C4 alkyl acrylate, methyl acrylate, ethyl acrylate, tert-butyl acrylate and the methacrylate derivatives of these); acrylate esters grafted onto a polyalkylene glycol such as polyethylene glycol (e.g., poly(ethyleneglycol)acrylate); hydroxyesters acrylate (e.g., hydroxyethyl methacrylate); acrylamides, methacrylamides which may or may not be substituted on the nitrogen by lower alkyl groups (C1-C4); N-alkylated acrylamide (e.g., N-tertbutylacrylamide); hydroxyalkylated acrylamide; amino alkylated acrylamide (e.g., dimethyl amino propyl methacrylamide); alkylacrylamine (e.g., tert-butylamino-ethyl methacrylate, dimethyl aminoethyl methacrylate); alkylether acrylate (e.g., 2-ethoxyethyl acrylate); monoethylenic monomer such as ethylene, styrene; vinyl esters (e.g., vinyl acetate or vinyl propionate, vinyl tert-butyl-benzoate; vinyl esters grafted onto a polyalkylene glycol such as polyethylene glycol; vinyl ether; vinyl halides; phenylvinyl derivatives; allyl esters or methallyl esters; vinyllactams such as vinylpyrrolidone or vinylcapro lactam; alkyl maleimide, hydroxyalkyl maleimide (e.g., Ethyl/Ethanol Maleimide). When present the anhydride functions of these polymers can optionally be monoesterified or monoamidated.

The anionic hairstyling polymers can be selected from: copolymers derived from acrylic acid such as the acrylic acid/ethylacrylate/N-tert-butylacrylamide terpolymer such as that sold as Ultrahold 8 by BASF®; Octylacrylamide/Acrylates/Butylaminoethyl/Methacrylate Copolymer such as that sold as Amphomer® by Akzo Nobel®; methacrylic acid/ester acrylate/ester methacrylate such that as sold as Balance® CR by Akzo Nobel®; Octylacrylamide/Acrylates/Butylaminoethyl Methacrylate Copolymer such as that sold as Balance® 47 by Akzo Nobel®; methacrylic acid/hydroxyethylmethacrylate/various acrylate esters such as that known as Acudyne™ sold 1000 by Dow® Chemical; acrylates/hydroxyethylmethacrylate such as that sold as Acudyne™ 180 by Dow® Chemical; methacrylic acid/hydroxyethylmethacrylate/various acrylate esters such as that sold as Acudyne™ DHR by Dow® Chemical; n-butyl methacrylate/methacrylic acid/ethyl acrylate copolymer such as that sold as Tilamar® Fix A-1000 by DSM®; copolymers derived from crotonic acid, such as vinyl acetate/vinyl tertbutylbenzoate/crotonic acid terpolymers and the crotonic acid/vinyl acetate/vinyl neododecanoate terpolymers such as that sold as Resyn™ 28-2930 by Akzo Nobel®. Hairstyling polymers derived from sulfonic acid can include: sodium polystyrene sulfonate sold as Flexan® 130 by Ashland™; sulfopolyester (also known as Polyester-5) such as that sold as Eastman AQ 48 by Eastman; sulfopolyester (also known as Polyester-5) such as that sold as Eastman AQ S38 by Eastman; sulfopolyester (also known as Polyester-5) such as that sold as Eastman AQ 55 by Eastman. The anionic hairstyling polymers can be selected from: copolymers derived from acrylic acid such as the acrylic acid/ethylacrylate/N-tert-butylacrylamide terpolymers (such as that sold as Ultrahold® 8 by BASF®); Octylacrylamide/Acrylates/Butylaminoethyl/Methacrylate Copolymer such as that sold as Amphomer; methacrylic acid/ester acrylate/ester methacrylate such as that sold as Balance® CR by Akzo Nobel®; Octylacrylamide/Acrylates/Butylaminoethyl Methacrylate Copolymer such as that sold as Balance® 47 by Akzo Nobel®; methacrylic acid/hydroxyethylmethacrylate/various acrylate esters such as that known as Acudyne® 1000 sold by Dow® Chemical; acrylates/hydroxyethylmethacrylate such as that sold as Acudyne® 180 by Dow® Chemical; methacrylic acid/hydroxyethylmethacrylate/various acrylate esters such as that sold as Acudyne® DHR by Dow® Chemical; n-butyl methacrylate/methacrylic acid/ethyl acrylate copolymer such as that sold as Tilamar® Fix A-1000 by DSM®; copolymers derived from crotonic acid, such as vinyl acetate/vinyl tertbutylbenzoate/crotonic acid terpolymers and the crotonic acid/vinyl acetate/vinyl neododecanoate terpolymers such as that sold as Resyn™ 282930 by Akzo Nobel®. Hairstyling polymers derived from styrene sulfonic acid can include: sodium polystyrene sulfonate sold as Flexan® 130 by Ashland™; sulfopolyester (also known as Polyester-5) such as that sold as Eastman AQ 48 by Eastman; sulfopolyester (also known as Polyester-5) such as that sold as Eastman AQ S38 by Eastman; sulfopolyester (also known as Polyester-5) such as that sold as Eastman AQ 55 by Eastman.

The hairstyling polymer can be an anionic hairstyling polymer, and wherein the anionic hairstyling polymer is selected from: copolymers derived from acrylic acid such as the acrylic acid/ethylacrylate/N-tert-butylacrylamide terpolymers; Octylacrylamide/Acrylates/Butylaminoethyl/Methacrylate Copolymers; methacrylic acid/ester acrylate/ester methacrylates; Octylacrylamide/Acrylates/Butylaminoethyl Methacrylate Copolymer; methacrylic acid/hydroxyethylmethacrylate/various acrylate esters; acrylates/hydroxyethylmethacrylate; methacrylic acid/hydroxyethylmethacrylate/various acrylate esters; n-butyl methacrylate/methacrylic acid/ethyl acrylate copolymers; copolymers derived from crotonic acid, such as vinyl acetate/vinyl tertbutylbenzoate/crotonic acid terpolymers; and the crotonic acid/vinyl acetate/vinyl neododecanoate terpolymers; and mixtures thereof.

The hairstyling polymer can be a polyurethane dispersed or dissolved in solvent (e.g., water, ethanol, or another alcohol). Such polyurethanes can include those such as adipic acid, 1-6 hexandiol, neopentyl glycol, isophorone diisocyanate, isophorone diamine, N-(2-aminoethyl)-3-aminoethanesulphonic acid, sodium salt (also known as Polyurethane-48) such as that sold as Baycusan® C1008 by Bayer®; and such as isophorone diisocyanate, dimethylol propionic acid, 4,4-isopropylidenediphenol/propylene oxide/ethylene oxide (also known as Polyurethene-14) such as that sold as a mixture under the name of DynamX® H20 by Akzo Nobel®.

The hairstyling polymer can be a nonionic hairstyling polymer or a mixture of nonionic hairstyling polymers such as Luviskol® VA 64 from BASF® and PVP K30 from Ashland). The non-ionic hairstyling polymer can be a water-soluble natural polymer such as hydroxyalkylcelluloses (e.g., hydroxymethyl-, hydroxyethyl- or hydroxypropylcelluloses) and starches.

The hairstyling polymer can be an amphoteric hairstyling polymer or a mixture of amphoteric hairstyling polymers such as Diaformer® Z 731 N from Clariant®.

Some non-limiting examples of hairstyling polymers can be found in co-pending U.S. Ser. No. 17/874,600, filed by Brown, et al., on Jul. 27, 2022.

The hairspray composition can be substantially free of water-insoluble and/or water immiscible polymers and/or alcohol-insoluble polymers. Polymers of high molecular weight (e.g., >200,000 g/mol) may be avoided or only used at very low levels so that the hairspray composition does not exceed the desired viscosity. The hairspray composition may be substantially free of a polymer comprising at least two long hydrophobic grafts (e.g., linear fatty chains of 10 carbons or more). Such polymers with such grafts can lead to associative interactions in the hairspray composition which can drive viscosity up without contributing to the strength of the film delivered to the hair.

### Ethanol-Based Hairspray

An ethanol-based hairspray composition may include an alcohol solvent present at 50% to 99.9% alcohol (e.g., 60% to 97%, 70% to 95%, or 80% to 95% ethanol, isopropanol or), by weight of the hairspray compositions. Some non-limiting examples of alcohol solvents include ethanol, n-propanol, isopropanol, and combinations thereof. The hairspray polymer used in the composition should generally be soluble in the alcohol solvent, but need not necessarily be so.

The hairspray composition may further comprise other additional solvents, including water, provided that such additional solvents are chemically and physically compatible with the ingredients of the composition and that it does not substantially and unduly impair product performance. Some non-limiting formulation examples and additional ingredients that can be used in an ethanol-based hairspray can be found in WO1998/05379.

### Ethanol-Free Hairspray

Some consumers may prefer an alcohol/ethanol-free or very low alcohol/ethanol hairspray because they can have a purer fragrance (in view of the absence of an alcohol smell), less observed hair dryness and reduced brittleness effects to the hair, and consumers may perceive them to be more environmentally friendly and/or healthier to use. Ethanol-free hairspray compositions herein contain less than 2% alcohol/ethanol, (e.g., less than 1%, 0.5%, or even less than 0.25% alcohol/ethanol), by weight of the hairspray composition. In some embodiments, the hairspray composition contains 0% ethanol or alcohol. The hairspray polymer used in the composition should generally be water soluble, but need not necessarily be so.

The hairspray composition can include 30% to 99% water (e.g., 60% to 98% water, 70% to 97% water, 80% to 96%, or 85% to 96%), by weight of the hairspray composition. The water can provide a solvent for the hairstyling polymer and other ingredients in the hairspray composition. It may be desirable to use ingredients for the hairspray composition that are water soluble.

### Compressed Gas Propellant

The compressed gas propellant can include gases such as nitrogen, air, carbon dioxide, nitrous oxide, and other inert gases.

### Optional Ingredients

The hairspray composition can include a panthenol compound and/or a silicone compound. The panthenol compound may be selected from the group consisting of: panthenol, a pantothenic acid derivative, and mixtures thereof. The panthenol compound can be selected from the group consisting of: D-panthenol ([R]-2,4-dihydroxy-N-[3-15-(hydroxypropyl)]-3,3-dimethylbutamide), D/L-panthenol, pantothenic acids and their salts, panthenyl triacetate, royal jelly, panthetine, pantotheine, panthenyl ethyl ether, pangamic acid, pantoyl lactose, Vitamin B complex, and mixtures thereof. The panthenol compound can be useful in view of providing excellent hair look and feel benefits. The hairspray composition may comprise 0.1% to 0.6% (e.g., 0.1% to 0.3%) of a panthenol compound, by weight of the hairspray composition. The hairspray composition can include a silicone compound. The silicone can be useful because it gives a smoother feel and also shine to the hair. The silicone compound can be a dimethicone compound. In The silicone compound can be a PEG dimethicone, for example PEG-12 dimethicone.

The hairspray composition may further include a surfactant present at 1% or less (e.g., 0.6% or less, 0.4% or less, or 0.3% or less), by weight of the hairspray composition. The surfactant may be selected from the group consisting of cationic surfactants, non-ionic surfactants, anionic surfactants, and mixtures thereof.

The hairspray composition can include a neutralizer. Suitable neutralizers may include potassium hydroxide, sodium hydroxide, triisopropanolamine (TIPA), 2-aminobutanol, 2-aminomethyl propanol (AMP), aminoethylpropandiol, dimethyl stearamine (Armeen 18 D), sodium silicate, tetrahydroxypropyl ethylenediamine (Neutrol® TE), ammonia (NH3), triethanolamine, trimethylamine (Tris AminoUltra), aminomethylpropandiol (AMPD). The neutralising agent can be 2-aminobutanol, ammonia, or 2-aminomethyl propanol.

The hairspray composition may include at least one preservative. The preservative may be present in an amount of less than 1.5%, or 0% to 1%, or 0.01% to 1%, by weight of the hairspray composition.

The hairspray composition may further include a perfume or fragrance. It may be desirable to limit the amount of perfume or fragrance to 0.5% (e.g., 0% to 0.4% or 0.03% to 0.3%), by weight of the hairspray composition.

The hairspray composition can include vitamins, amino acids and preservatives.

Additional information on hairspray compositions and aerosol spray dispensers is found in U.S. Pat. Nos. 9,986,809, 10,131,488, and 10,426,979.

### Test Methods

### Spray Rate

The spray rate of may be determined following ASTM D 3069-94, “Standard Test Method for Delivery Rate of Aerosol Products.” In this test, the delivery rate of the product is determined by measuring the mass lost in a given time period. This correlates with the quantity of material expelled though the valve and actuator combination in a given time period. In this case, the can is tested at room temperature (at 21° C.) and a duration of 2 sec to 10 sec for the actuation time. The delivery rate is then determined by the equation:

Spray Rate (g/sec)=Mass loss (g)/Actuation time (sec)

If the spray rate is greater than 0.45 g/sec, then the on-hair drying time may be too long for consumer satisfaction. This is unique for the ethanol-free hairsprays described herein as compared to traditional ethanol-based hairsprays, typically traditional hairsprays containing volatile alcohol have delivery rates between 0.55 g/sec and 0.85 g/sec. Delivery rate can typically be adjusted by altering the pressure inside the container (increased pressure correlates with faster delivery rate) and/or the orifices in the spraying device, such as the orifices in the nozzle, orifices in the valve, and the inner diameter of the dip tube.

### Particle Size Distribution (Dv50)

Dv50 is the maximum particle size diameter below which 50% of the sample volume possesses. Dv50 is sometimes referred to as the median particle size by volume. Dv90 is the maximum particle size diameter below which 90% of the sample volume possesses.

The droplet size through the life of the can may be important as it impacts the dry feel, dry time and hold performance of the hairspray. Smaller droplets dry faster. More small droplets feel less wet than fewer large droplets. For hold, a larger number of smaller droplets give more surface area coverage to provide an even coating of hair welds. If the droplets are too small they will not bridge hair together in weld points and won't hold. This may be important for alcohol-free hairsprays that do not have the fast evaporation drying advantage of ethanol formulas. Ethanol hairsprays have a much broader working droplet size range, e.g., 30-130 um. An ethanol-free hairspray below 40 um Dv50 may have holding problems. An ethanol-free hairspray above 80 um Dv50 may have a noticeably slow drying time and initial wet hair feel. The Dv90 represents, though much fewer, the largest droplets in the spray. Dv90 values over 180 um that get larger, up to 400 um, over the life of the can may result in sprays that appear visually uneven, with larger and sputtering droplets. These large drops make the spray less misty and even. This may result in clumping of hair where those large drops land. These hair clumps may make the end finished hair results have an unnatural hair feel that is difficult to run fingers or a brush through.

The average particle size distribution (Dv50) may be important in view of ejected composition drying time, which must be consumer acceptable. Indeed, a smaller average particle size distribution (Dv50) may be useful in that more particles have a higher surface area to volume ratio, which means a faster drying time. On the other hand, a too low average particle size distribution (Dv50) may mean that not enough hairstyling polymer is provided to the hair to provide spot welds.

A Malvern Spraytec™ instrument is used to measure the particle size distribution. The Malvern Spraytec™ instrument uses the technique of laser diffraction for measurement of the size of the spray particles. The intensity of light scattered as a laser beam passes through a spray is measured. This data is then analyzed to calculate the size of the particles that created the scattering pattern. A Malvern Spraytec™ 2000 is used according to the manufacturer's instructions. Test samples have a temperature between 20° C. to 22° C.

### Spray Diameter

The spray pattern through the life of the can may be important as it impacts the dry feel, dry time and hold performance of the hairspray. A smaller spray diameter, or more localized spray, can cause hair to feel wetter, take longer to dry, and provide more hold due to more and larger welds between hairs. A larger spray diameter, or mistier spray, can result in areas where droplets do not bridge hair together in weld points and won't hold, which can be undesirable. Given the general size of the human head and the distance one can comfortably hold the container away from the head, a spray diameter of 2 inches to 6 inches is desirable.

Spray diameter is measured using thermal sensitive paper mounted on a rigid test stand. Test samples are equilibrated to 20 to 24° C. The test can is placed perpendicular +/−10° to the paper at a distance of 6 inches. After spray is completed, the test paper is scanned and analyzed with imaging software. The outside diameter of the spray is calculated by

$$Diameter = 2 * ( “ area ” / 3.141593 ) ⋀ 0.5$$

Where “area” is the cell location in the imaging software containing the outside diameter area

### Viscosity

Kinematic viscosity may be measured with a Ubbelohde tube viscometer. Kinematic viscosity is a measure of the resistance to flow of a fluid, equal to its absolute viscosity divided by its density. The SI unit of kinematic viscosity is m²·s⁻¹. The cgs (centimeters/grams/seconds) physical unit for kinematic viscosity is the stokes (St), which can be expressed in terms of centistokes (cSt). 1 cSt=1 mm²·s⁻¹=10⁻⁶ m²·s⁻¹. Water at 20° C. has a kinematic viscosity of 1 cSt. A Ubbelohde tube is a viscometer for measurement of kinematic viscosity of transparent Newtonian liquids by suspended level principle as described in ASTM D 445 and D 446, and ISO 3104 and 3105. For the Ubbelohde tube measure, there is no temperature effect on results, for other kinematic viscometers, temperatures different from specified test range can affect result. Herein, measurements were taken at a temperature of 20° C.+/−0.1° C. This method can be used to measure 0.6 cSt to 100 cSt. For instructions for the use of the Ubbelohde viscometer see ASTM D 445. Use Ubbelohde tube size 0 C for viscosities from 0.6 to 3 cSt at 20° C.+/−0.1° C. Use Ubbelohde tube size 1 for viscosities from 2 to 10 cSt at 20° C.+/−0.1° C. ASTM D445 is the “Standard Test Method for Kinematic Viscosity of Transparent and Opaque Liquids”. ASTM D446 is “Specifications and Operating Instructions for Glass Capillary Kinematic Viscometers”.

### EXAMPLES

Aerosol dispensers were filled with either an ethanol-free hairspray (i.e. water-based hairspray) or an ethanol-based hairspray and 60% nitrogen propellant. Each experimental aerosol dispenser had an aerosol assembly that included one of four Salvalco Eco-Valves® that were identified by the insert color (pink, green, red, blue, clear or yellow). Each Salvalco Eco-Valve® appears to have different configurations that can include varying the exit orifice thickness and diameter and/or varying the number, height, and width of the channels tangential to the orifice. It is believed, without being limited by theory, that different spraying device configurations influence the spray properties.

The spray properties (e.g., spray rate (g/s) and droplet size (μm)) of the aerosol dispensers with the Salvalco Eco-Valves® were compared to a control (Pantene® Pro-V Airspray Flexible Hold Hairspray) throughout the life of the dispenser. The can was filled to 40-60% nitrogen at 135 PSIG. The cans were tested from 100% product (full can) to when 25% of the product was left in the can. Initial values are obtained when the container is full (i.e., when 100% of the composition is present), and final values are obtained when approximately 25% of the hairspray composition remains. The test results for the ethanol-based hairspray composition are summarized in Table 2A. The test results for the ethanol-free hairspray composition are summarized in Table 2B.

TABLE 2A

| | | | Spray | Spray | | | | |
|---|---|---|---|---|---|---|---|---|
| | | | Rate | Rate | Dv50 | | Spray | Spray |
| | Insert | Insert | initial | final | initial | Dv50 | Diameter | Diameter |
| | Color | Reference | (g/s) | (g/s) | (μm) | final | initial (cm) | final (cm) |
| Ethanol- | Green | LP05 | 0.39 | 0.36 | 70 | 82.8 | 7.6 | 7.0 |
| based | Pink | LP06 | 0.46 | 0.38 | 70 | 87.6 | 8.9 | 7.6 |
| hairspray | Red | LQ05 | 1.20 | 1.02 | 79 | 90.6 | 16.5 | 14.0 |
| (Table 7, | Blue | LQ06 | 1.02 | 0.93 | 80 | 93.2 | 10.2 | 8.9 |
| Ex. J) | Clear | LP11 | 0.97 | 0.89 | 78 | 61.5 | 10.2 | 9.5 |
| | Yellow | LP09 | 0.67 | 0.60 | 76 | 69.7 | 8.9 | 7.6 |

TABLE 2B

| | | | | | Meets overall |
|---|---|---|---|---|---|
| | | | Dv50 | Is Dv50 final | target spray |
| | Insert | Insert | initial | within 20 μm | properties in |
| | Color | Reference | (μm) | of Dv50 initial? | Table 1? |
| Ethanol-free | Green | LP05 | 64 | Yes | Yes |
| Hairspray (Table | Pink | LP06 | 63 | Yes | Yes |
| 5, Ex. A) | Red | LQ05 | 72 | No | No |
| | Blue | LQ06 | 66 | No | No |
| | Clear | LP11 | 84 | Yes | No |
| | Yellow | LP09 | 82 | Yes | No |

In the examples in Table 3 and Table 4, below, an aerosol container was filled with nitrogen gas and a hairspray composition. The aerosol container included the Salavaco Eco-Valve with the green insert. The hairsprays in Table 3 contain twice the concentration of actives and a higher viscosity, as compared to the paired example in Table 4.

The examples in Table 3 and Table 4, were tested to see if they met the properties described in Table 1, herein. The spray properties were maintained if the initial spray rate and Dv50 compared to the spray rate and Dv50 when 25% of the hairspray composition remained in the container varied by less than 10% and no more than 20 microns, respectively.

TABLE 3
Spray Rate and Dv50 of compositions with a high concentration of actives

| | | 40% N₂ Fill | | 60% N₂ Fill | |
|---|---|---|---|---|---|
| | | Spray Rate | Dv50 | Spray Rate | Dv50 |
| | | (g/s) | (μm) | (g/s) | (μm) |
| Ethanol-based | initial | 0.44 | 118 | 0.49 | 103 |
| Hairspray | 25% composition | 0.38 | 141 | 0.48 | 108 |
| (Table 7, Ex. JJ) | remaining | | | | |
| Ethanol-free | initial | 0.49 | 115 | 0.50 | 85 |
| Hairspray | 25% composition | 0.43 | 115 | 0.47 | 93 |
| (Table 5, Ex. AA) | remaining | | | | |

None of the examples tested in Table 3 meet all of the success criteria outlined herein. For the ethanol-based hairspray (Example JJ in Table 7, below) combined with 40% and 60% nitrogen propellant, the spray rate was consumer acceptable, however the Dv50 average particle size was too large to be a consumer acceptable hairspray product.

For the ethanol-free hairspray (Example AA in Table 5, below), the spray rate was consumer acceptable for the examples with 40% and 60% nitrogen propellant. However, the particle size for these examples was outside the success criteria. For the 40% nitrogen propellant, the particle size was too large and for the 60% nitrogen propellant at both test points. For the 20% nitrogen propellant, the initial particle size was consumer acceptable, however when 25% of the composition was remaining, the average particle size was too large to be consumer acceptable.

Examples AA and JJ, which were tested in Table 3, had a concentration of actives similar to current products that use a hydrocarbon propellant. It was found that since the nitrogen propellant did not liquify, like a hydrocarbon propellant, a less concentrated hairspray composition could be used, while still providing excellent styling benefits. Additionally, since the Dv50 particle size for 40% nitrogen fill was too high for both ethanol-based and ethanol-free hairsprays and the Dv50 particle size for 60% was closer to the preferred range for both the initial and 25% of the product remaining, it was determined that 60% nitrogen would be used for the lower concentration formulas.

TABLE 4
Spray Rate and Dv50 Hairsprays with
a Lower Concentration of Actives

60% N₂ Fill

| | | Spray Rate, | Dv50 |
|---|---|---|---|
| | | (g/s) | (μm) |
| Ethanol-based | initial | 0.42 | 70 |
| Hairspray | 25% composition | 0.39 | 75 |
| (Table 7, Ex. J) | remaining | | |
| Ethanol-free | initial | 0.44 | 64 |
| Hairspray | 25% composition | 0.42 | 63 |
| (Table 5, Ex. A) | remaining | | |

Examples J (Table 7, below) and A (Table 5, below) that were tested in Table 4 have half of the active level as compared to Examples JJ and AA, which were tested in Table 3. Both the ethanol-based hairspray (Example J) and the ethanol-free hairspray (Example A) have spray rates and Dv50 average particle size that are consumer acceptable at the initial spray and when 25% of the hairspray composition is remaining in the can.

The examples in Table 5, Table 6, and Table 7 can be made using a conventional method of making hairspray compositions and products.

TABLE 5
Examples A-D Ethanol-Free Hairspray Compositions (active level %)

| | A | AA | B | C | D |
|---|---|---|---|---|---|
| Acrylates Copolymer ¹ | 4.20% | 7.31% | 4.50% | 2.90% | 4.50% |
| Polyurethane-14/AMP- | 1.10% | 1.83% | 1.30% | 2.90% | — |
| acrylates polymer blend ² | | | | | |
| Octylacrylamide/Acrylates/But | 0.60% | 1.1% | — | — | — |
| ylaminoethyl/Methacrylate | | | | | |
| Copolymer ⁴ | | | | | |
| Vinylpyrrolidone/ Vinylacetate | — | — | — | — | — |
| Copolymer ⁵ | | | | | |
| Methacrylic acid/ | — | — | 0.60% | 1.50% | — |
| hydroxyethylmethacrylate/vario | | | | | |
| us acrylate esters ³ | | | | | |
| Polyquaternium-16 ⁶ | — | — | — | — | — |
| Chitosan ⁷ | — | — | — | — | — |
| Hydroxyethylcellulose | — | — | — | — | — |
| dimethyldiallyammonium | | | | | |
| chloride[PQ4] ⁸ | | | | | |
| 2-aminomethyl propanol | 0.70% | 1.20% | 0.36% | 0.34% | 0.48% |
| (AMP) | | | | | |
| Potassium Hydroxide | — | — | 0.48% | 0.45% | — |
| Fragrance | 0.08% | 0.16% | 0.10% | 0.10% | 0.10% |
| Dehyquart A-CA /Detex | 0.1% | 0.2% | 0.2% | 0.2% | — |
| (cationic surfactant) | | | | | |
| Dehydol LS 4 Deo N (Non- | 0.05% | 0.1% | 0.05% | 0.05% | — |
| ionic surfactant) | | | | | |
| PEG-12 dimethicone | 0.13% | 0.26% | — | 0.13% | — |
| Disodium EDTA | 0.11% | 0.22% | 0.11% | 0.11% | 0.11% |
| Phenoxyethanol | 0.50% | 1.00% | 0.50% | 0.50% | 0.50% |
| Methylparaben | 0.10% | 0.20% | 0.10% | 0.10% | 0.10% |
| Water | QS | QS | QS | QS | QS |

KEY:
¹ = Balance ® CR Polymer;
² = DynamX H20;
³ = Acudyne 1000;
⁴ = Amphomer;
⁵ = Luviskol VA64;
⁶ = Luviquat FC550;
⁷ = Hydagen ® HCMF;
⁸ = Celquat L-200.

TABLE 6
Examples E-I Ethanol-Free Hairspray Compositions (active level %)

| | E | F | G | H | I |
|---|---|---|---|---|---|
| Acrylates Copolymer ¹ | 1.60% | — | — | — | — |
| Polyurethane-14/AMP- | 2.00% | — | — | — | — |
| acrylates polymer blend ² | | | | | |
| Octylacrylamide/Acrylates/But | — | 1.7% | — | — | — |
| ylaminoethyl/Methacrylate | | | | | |
| Copolymer ⁴ | | | | | |
| Vinylpyrrolidone/Vinylacetate | — | — | 7.00% | — | 5.75% |
| Copolymer ⁵ | | | | | |
| Methacrylic | 3.00% | — | — | — | — |
| acid/hydroxyethylmethacrylate/ | | | | | |
| various acrylate esters ³ | | | | | |
| Polyquaternium-16 ⁶ | — | — | — | 2.5% | — |
| Chitosan ⁷ | — | — | — | 0.75% | — |
| Hydroxyethylcellulose | — | — | — | — | 0.83% |
| dimethyldiallyammonium | | | | | |
| chloride[PQ4] ⁸ | | | | | |
| 2-aminomethyl propanol | — | 0.32% | — | — | — |
| (AMP) | | | | | |
| Potassium Hydroxide | 0.98% | — | — | 0.40% | — |
| Fragrance | 0.10% | 0.35% | 0.18% | 0.15% | 0.2% |
| Dehyquart A-CA /Detex | — | — | — | 0.2% | 0.2% |
| (cationic surfactant) | | | | | |
| Dehydol LS 4 Deo N (Non- | — | — | 0.10% | — | 0.10% |
| ionic surfactant) | | | | | |
| PEG-12 dimethicone | — | — | — | — | — |
| Disodium EDTA | 0.11% | 0.11% | 0.11% | 0.11% | 0.11% |
| Phenoxyethanol | 0.50% | 0.50% | 0.50% | 0.50% | 0.50% |
| Methylparaben | 0.10% | 0.10% | 0.10% | 0.10% | 0.10% |
| Water | QS | QS | QS | QS | QS |

KEY:
¹ = Balance ® CR Polymer;
² = DynamX H20;
³ = Acudyne 1000;
⁴ = Amphomer;
⁵ = Luviskol VA64;
⁶ = Luviquat FC550;
⁷ = Hydagen ® HCMF;
⁸ = Celquat L-200.

TABLE 7
Examples J-K Ethanol-based Finishing Hairspray

| | J | JJ | K | L |
|---|---|---|---|---|
| VA/Crotoantes/Vinyl | 2.16% | 3.61% | — | — |
| Neodecanoate Copolymer ¹ | | | | |
| Octylacrylamide Acrylates | 1.94% | 3.23% | — | — |
| Copolymer² | | | | |
| Octylacrylamide/Acrylates/ | — | — | 3.25% | 4.50% |
| Butylaminoethyl/Methacrylate | | | | |
| Copolymer ³ | | | | |
| Vinylpyrrolidone/Vinylacetate | — | — | — | — |
| Copolymer ⁵ | | | | |
| Water | 4.37% | 7.20% | 0.15% | 0.15% |
| 2-aminomethyl propanol (AMP) | 0.55% | 0.92% | 0.72% | 0.98% |
| Tapioca Starch | — | — | 1.50% | — |
| Silica | — | — | 0.25% | — |
| Polysorbate-80 | 0.13% | 0.22% | — | — |
| Ammonium Benzoate | 0.20% | 0.33% | — | — |
| Monoethanolamine Borate | 0.20% | 0.33% | | |
| Fragrance | 0.27% | 0.45% | 0.10% | 0.10% |
| Triethyl Citrate | — | — | 0.15% | — |
| Ethanol | QS | QS | QS | QS |

KEY:
¹ = Resyn 28-2930;
²= Amphomer;
³ = Balance 47;

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol hairspray product, comprising:
a) a pressurizable container comprising a container wall that encloses a reservoir for storing a compressed gas propellant and a hairspray composition, the hairspray composition comprising:
i) about 30% to about 98.5% of a solvent, by weight of the hairspray composition, and
ii) about 1.5% to about 8% hairstyling polymer, by weight of the hairspray composition, wherein the hairstyling polymer is soluble in the solvent;
b) a spraying device clinched onto the container for dispensing the hairspray composition from the reservoir of the container, wherein the spraying device comprises:
i) a valve assembly comprising a housing with internal walls defining a valve chamber, wherein the valve chamber comprises a liquid inlet in fluid communication with the hairspray composition in the reservoir, and a gas inlet in fluid communication with the compressed gas propellant in the reservoir,
ii) a valve stem comprising a proximal end and a distal end, wherein the proximal end is received in the valve chamber and the distal end projects through a sealed opening in the valve chamber, the valve stem further comprising an outlet flow conduit with an outlet aperture at the distal end, a first stem inlet for receiving liquid and a second stem inlet for receiving gas,
wherein the housing includes a lip around a perimeter of the valve stem to form a seal around the perimeter of the valve stem,
wherein the valve chamber liquid inlet is proximal of the lip and the valve chamber gas inlet is distal of the lip, and
wherein the valve stem is moveable between:
a closed position, wherein the first stem inlet is distal of the lip and the second stem inlet is distal of the sealed opening in the valve chamber, such that the first stem inlet is not in fluid communication with the valve chamber liquid inlet and the second stem inlet is not in fluid communication with the valve chamber gas inlet, and
an open position, wherein the first stem inlet is proximal of the lip and in fluid communication with the valve chamber liquid inlet, and the second stem inlet is proximal of the sealed opening in the valve chamber and at least partially distal of the lip and in fluid communication with the valve chamber gas inlet, whereby a bubble laden flow is created in the outlet flow conduit;
c) an actuator joined to the spraying device; and
d) an overcap that covers the actuator and a top portion of the container.

2. The aerosol hairspray product of claim 1, wherein the actuator comprises an outlet orifice having an outlet orifice area and the valve stem comprises an orifice having a valve stem orifice area, and wherein a ratio of the valve stem orifice area to the actuator outlet orifice area is less than about 10, preferably less than 8 and more preferably less than 5.

3. The aerosol hairspray product of claim 2, wherein the ratio of the valve stem orifice area to the actuator outlet orifice area is less than about 5.

4. The aerosol hairspray product of claim 1, wherein the valve assembly is vertically oriented, and the actuator is seated on a vertically oriented valve system.

5. The aerosol hairspray product of claim 1, wherein the solvent is water and the hairspray composition is free of alcohol.

6. The aerosol hairspray product of claim 1, wherein the compressed gas propellant comprises nitrogen.

7. The aerosol hairspray product of claim 1, further comprising about 5.5% to about 8% of an anionic hairstyling polymer comprising a hairstyling polymer derived from acrylic acid, crotonic acid or combination thereof.

8. The aerosol hairspray product of claim 7, wherein the anionic hairstyling polymer comprises an acrylic acid/ethylacrylate/N-tert-butylacrylamide terpolymer, an acrylates/hydroxyesters acrylates copolymer of butyl acrylate, a methyl methacrylate, methacrylic acid, an ethyl acrylate and hydroxyethyl methacrylate, polyurethane-14/AMP-acrylates copolymer blend, an octylacrylamide/acrylates/butylaminoethyl/methacrylate copolymer, a methacrylic acid/ester acrylate/ester methacrylate copolymer, an acrylates/hydroxyethylmethacrylate copolymer, a methacrylic acid/hydroxyethylmethacrylate/acrylate ester copolymer; an N-butyl methacrylate/methacrylic acid/ethyl acrylate copolymers or a combination thereof.

9. The aerosol hairspray product of claim 7, wherein the anionic hairstyling polymer comprises a vinyl acetate/vinyl tertbutylbenzoate/crotonic acid terpolymer, a crotonic acid/vinyl acetate/vinyl neododecanoate terpolymer or a combination thereof.

10. The aerosol hairspray product of claim 1, wherein the hairspray product has a spray rate of 0.3 g/sec to 1.0 g/sec according to the Spray Rate test.

11. The aerosol hairspray product of claim 10, wherein the spray rate of the hair spray product varies by no more than 30% from when the container contains 100% of the hairspray composition to when the container contains 25% of the hairspray composition.

12. The aerosol hairspray product of claim 1, wherein the hairspray product has a Dv50 of 40 μm to 90 μm, according to the Particle Size Distribution test.

13. The aerosol hairspray product of claim 12, wherein the Dv50 varies by no more than 20 μm from when the container contains 100% of the hairspray composition to when the container contains 25% of the hairspray composition.

14. The aerosol hairspray product of claim 1, wherein the hairspray product has a spray diameter of 5 cm to 15.25 cm.

15. The aerosol hairspray product of claim 14, wherein the spray diameter varies by no more than 30% from when the container contains 100% of the hairspray composition to when the container contains 25% of the hairspray composition.

16. The aerosol hairspray product of claim 1, wherein at least one of the container, the spraying device, the actuator and the overcap comprise 10% or more of a material selected from recycled materials, bio-based plastics and combinations of these.

17. A method of styling hair, comprising:
a) providing the aerosol hairspray product of claim 1; and
b) applying the hairspray composition to a target portion of hair wherein a styling benefit is desired.
